(19) United States
(12) Patent Application Publication
(10) Pub. No.: US 2016/0310032 A1
(43) Pub. Date: Oct. 27, 2016

(54) FABRIC SENSOR, METHOD OF MAKING THE FABRIC SENSOR, AND APPLICATIONS THEREOF

(71) Applicant: THE UNIVERSITY OF CONNECTICUT, Farmington, CT (US)

(72) Inventors: Gregory Allen Sotzing, Storrs, CT (US); Yang Cao, Glastonbury, CT (US)

(21) Appl. No.: 15/135,895

(22) Filed: Apr. 22, 2016

Related U.S. Application Data
(60) Provisional application No. 62/151,623, filed on Apr. 23, 2015.

(51) Int. Cl. A61B 5/0408; A61B 5/08; A61B 5/11; A61B 5/00

(52) U.S. Cl. CPC A61B 5/04085; A61B 5/4818; A61B 5/0809; A61B 5/6802; A61B 5/1118; B29L 2031/34

(57) ABSTRACT

A cardio-respiratory sensor is described. The sensor includes at least two conductive electrodes; and an electroactive region disposed between and in contact with the at least two conductive electrodes. The conductive electrodes as well as the electroactive region can comprise fabric materials.

### CROSS-REFERENCE TO RELATED APPLICATIONS

This application claims the benefit of U.S. Provisional Application Ser. No. 62/151,623, filed Apr. 23, 2015, which is incorporated herein by reference in its entirety.

### TECHNICAL FIELD

This disclosure is generally related to sensors made of fabric material, methods of making the sensors, and applications of the sensors.

### BACKGROUND

Heart and respiratory rate monitoring has been an active area of interest in health care and sporting industry. Athletes and their trainers have been long using a variety of devices to monitor heart and respiratory rates during exercise and sports training. However, these monitoring devices suffer from a number of limitations.

Presently, in sporting industry heart rate is commonly monitored with accelerometers which measure heart rate based on stretch of a sensor. During exercise and sports, due to stretching or engaging of muscles physical fluctuations occur that disrupt the readings of such stretch based sensors. Therefore, such sensors are not ideal.

Conventional health monitoring systems rely on discrete devices, such as wrist module, watch, or hat, with integrated sensors for multi-vital signs sensing and monitoring. Also available are garments integrated with knitted-yarn-based sensors that use metallic electrodes and metallic wires for signal trans-conduction. Such health monitoring systems and sensors are typically bulky and often uncomfortable or even irritable to users. These disadvantages limit the wide-spread adoption of these “wearable” healthcare devices.

In view of the above challenges, it is noted that there is a need to provide new type of vital sign (heart and breathing rate) sensors. Such sensors should be free of metallic components, should not be bulky, and should not cause irritation to the users. This disclosure addresses the above described challenges.

### BRIEF DESCRIPTION OF EMBODIMENTS

One embodiment is a sensor, comprising at least two conductive electrodes; and an electroactive region disposed between and in contact with the at least two conductive electrodes.

Another embodiment is a method of making a sensor comprising stretching a fabric to create micro-voids in the fabric, subjected the fabric to electrical poling thereby forming an electroactive fabric, attaching the electroactive fabric to at least two conductive electrodes such that the electroactive fabric is disposed between and is in electrical contact with the at least two conductive electrodes.

These and other embodiments are described in detail below.

### BRIEF DESCRIPTION OF THE DRAWINGS

The following Figures are exemplary embodiments, which do not limit the claims.

FIG. 1 is a schematic diagram of the sensor.

FIG. 2A is a microscopy image showing cross-sectional view of an open pore electroactive fabric (expanded PTFE).

FIG. 2B is a microscopy image showing top view of an open pore electroactive fabric (expanded PTFE).

FIG. 3A is a microscopy image showing cross-sectional view of a closed pore electroactive fabric (propylene).

FIG. 3B is a microscopy image showing top view of a closed pore electroactive fabric (propylene).

FIG. 4 is a graph showing the D-E loop for open pore electroactive fabric (Expanded PTFE) revealed by a modified Sewyer-Tower tester, indicating ferroelectric hysteresis behavior.

FIG. 5 is a graph showing the D-E loop for closed pore electroactive fabric (propylene) revealed by a modified Sewyer-Tower tester, indicating ferroelectric hysteresis behavior.

FIG. 6A shows heart beat signal as recorded by a synthetic leather based sensor.

FIG. 6B shows respiration rate signal as recorded by a synthetic leather based sensor.

FIG. 7 shows respiratory rate and heart beat rate signals as recorded by a cardio-respiratory sensor using spandex electrodes (PEDOT-PSS coating).

### DETAILED DESCRIPTION OF THE INVENTION

The present inventors have created a sensor that is based on fabric electrodes and fabric sensing material. In other words, the sensor relies on fabric materials for sensing and signal transmission.

In one embodiment, the sensor does not include any metallic components. Such sensor may be referred to as an all-fabric sensor. The fabrics used in the sensor are entirely organic in composition, and can be readily integrated into textile based garment to provide vital signs monitoring without any compatibility issue. In addition, the sensor is self-powered and does not require external power supply like battery for sensing. The ease of integration and deployment make it ideal for cardiac, asthma, and sleep apnea patients monitoring, and for athletics performance and conditioning monitoring.

In one embodiment there is provided a sensor comprising at least two conductive electrodes; and an electroactive region disposed between and in contact with the at least two conductive electrodes.

In some embodiments, the electroactive region comprises at least one layer of an electroactive fabric. In some other embodiments, the electroactive region comprises at least two layers of an electroactive fabric. In some other embodiments, the electroactive region comprises three or more layers of an electroactive fabric.

In some embodiments, the electroactive region comprises a combination of two or more electroactive fabrics.

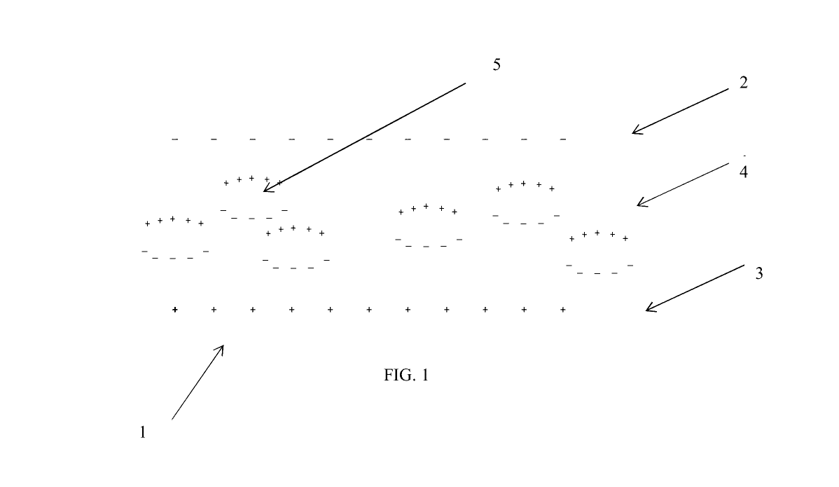

FIG. 1 shows a schematic diagram of an embodiment of the sensor. As shown in FIG. 1, the sensor comprises conductive electrodes 2 and 3, and an electroactive fabric 4. The electroactive fabric 4 is disposed between and is in contact with the conductive electrodes 2 and 3. The electroactive fabric 4 comprises polarized pores 5.

The conductive electrodes can be any suitable electrodes. The conductive electrodes can be metal electrodes or non-metallic electrodes. In one embodiment, the conductive electrodes are fabric electrodes.

In some embodiments, the conductive electrodes are formed from electrically conductive fibers. In some embodiments, the conductive electrodes comprise non-electrically conductive fibers that are coated with an electrically conductive material. The term “fiber” as used herein includes single filament and multi-filament fibers, i.e., fibers spun, woven, knitted, crocheted, knotted, pressed, plied, or the like from multiple filaments. No particular restriction is placed on the length of the conductive fiber, other than practical considerations based on manufacturing considerations and intended use. Similarly, no particular restriction is placed on the width (cross-sectional diameter) of the conductive fibers, other than those based on manufacturing and use considerations. The width of the fiber can be essentially constant, or vary along its length. For many purposes, the fibers can have a largest cross-sectional diameter of 2 nanometers and larger, for example up to 2 centimeters, specifically from 5 nanometers to 1 centimeter. In an embodiment, the fibers can have a largest cross-sectional diameter of 5 to 500 micrometers, more particularly, 5 to 200 micrometers, 5 to 100 micrometers, 10 to 100 micrometers, 20 to 80 micrometers, or 40 to 50 micrometers. In one embodiment, the conductive fiber has a largest circular diameter of 40 to 45 micrometers. Further, no restriction is placed on the cross-sectional shape of the conductive fiber, providing the desirable properties such as electrochromic behavior, flexibility, and/or stretchability are not adversely affected. For example, the conductive fiber can have a cross-sectional shape of a circle, ellipse, square, rectangle, or irregular shape.

When electrically conductive fibers are used, the fibers can comprise an electrically conductive material such as a metal, an electrically conductive organic material, or a combination thereof. Metals typically have a conductivity on the order of 10⁴ Siemens per centimeter (S/cm) or higher, while conductive organic materials typically have a conductivity on the order of 10⁻⁻¹ to 10³ S/cm.

Exemplary electrically conductive metals that can be formed into flexible fibers include silver, copper, gold, iron, aluminum, zinc, nickel, tin, and combinations comprising at least one of the foregoing metals. Iron and iron alloys such as stainless steel (an alloy of carbon, iron, and chromium) can be used. In one embodiment, the fibers consist essentially of a metal or metal alloy such as stainless steel. In another embodiment, the fiber consists of a metal or metal alloy such as stainless steel.

In some embodiments, the conductive electrodes are made of non-metallic materials. In other embodiments, the conductive electrodes are completely free of metals.

In some embodiments, the conductive electrodes comprise electrically conductive organic materials. Exemplary electrically conductive organic materials that can be formed into flexible fibers include conjugated polymers such as poly(thiophene), poly(pyrrole), poly(aniline), poly(acetylene), poly(p-phenylenevinylene) (PPV), poly(3,4-ethylenedioxythiophene):poly(styrene sulfonate)(PEDOT-PSS), and the like.

Electrically conductive fibers formed from non-conductive fibers that have been rendered electrically conductive can also be used. In one embodiment, a nonconductive fiber is coated with a layer of a conductive material. Exemplary nonconductive fibers include those known for use in the manufacture of fabrics, including natural materials (e.g., cotton, silk, and wool) and synthetic organic polymers (e.g., poly(amide) (nylon), poly(ethylene), poly(ester), poly(acrylic), polyurethane (spandex), poly(lactide), and the like). Specific fibers of this type include a nylon or spandex fiber. The above-described metal and organic polymer conductive materials can be used to coat the nonconductive fibers.

In one embodiment, nylon or spandex fiber is coated with PEDOT-PSS. The coated fibers can be used as a fiber as described herein, or at least two coated fibers can be woven, knitted, crocheted, knotted, pressed, or plied to form a multi-filament fiber. It is also possible to have multiple nonconductive fibers formed into a yarn, and then coated with a conductive material. This construction can be used as a fiber, or be woven, knitted, crocheted, knotted, pressed, or plied to form a multi-filament fiber.

Alternatively, a combination of electrically nonconductive and conductive fibers can be used to form an electrically conductive fiber. In another embodiment, one or more non-electrically conductive fibers are wrapped with an electrically conductive fiber, ribbon, or tape.

In some embodiments, any of the exemplary non-conductive fibers disclosed herein can be coated with an electrically conductive polymer, for example conjugated polymers such as poly(thiophene), poly(pyrrole), poly(aniline), poly(acetylene), poly(p-phenylenevinylene) (PPV), PEDOT-PSS, and the like. In a specific embodiment, a flexible, elastic fiber is coated with an electrically conductive material such as PEDOT-PSS, sulfonated polythieno[3,4-b]thiophene polystyrenesulfonate, the various poly(aniline)s (e.g., those sold by Enthone under the trade name ORMECON), and the like. In a specific embodiment, a nylon or spandex fiber is coated with PEDOT-PSS.

The electrically conductive fiber can be used in the form of a single fiber, a yarn, or a fabric. A “yarn” as used herein is a multi-fiber thread formed from two or more of the electrically conductive fibers by a variety of means, including but not limited to spinning, braiding, knitting, crocheting, knotting, pressing, and plying. The fabric can be woven (e.g., a mesh, twill, satin, basket, leno or mock leno weave) or nonwoven (e.g., a felt, wherein the fibers are entangled).

In some embodiments, the electroactive fabric are polymer-air composite films, with air filled pores in either closed-pore or open-pore structures ranging in size from a few microns to a few tens of microns. Electric charging of the polymer-air composite films under high voltage will induce electrical discharge of air within these micro-pores. The results incharge separation and permanent trapping of charges inside the polymer voids will form electroactive fabric with large piezoelectric coefficient in film thickness direction.

In some embodiments, the electroactive fabric comprises a closed pore fabric. In a closed-cell fabric, the gas forms discrete pockets, each completely surrounded by the solid material. A camping mat is an example of a closed-cell fabric, wherein the gas pockets are sealed from each other so the mat cannot soak up water.

In some embodiments, the electroactive fabric comprises an open-pore fabric. In an open-pore fabric, the gas pockets are connected with each other. A bath sponge is an example of an open-pore fabric, wherein water can easily flow through the entire structure, displacing the air. In some embodiments, the open pore electroactive fabric is selected from the group consisting of propylene, polyethylene, fluorinated ethylene propylene, and PTFE.

Suitable electroactive fabrics include polyolefins, such as polyethylene, polypropylene, fluoropolymers, such as tetrafluoroethylene, ethylenetetrafluoroethylene, fluororinated ethylene-propylene, containing pores preferable in a few micrometers to a few tens of micrometers through the film thickness direction.

The foregoing and other embodiments are further illustrated by the following examples, which are not intended to limit the effective scope of the claims. All parts and percentages in the examples and throughout the specification and claims are by weight of the final composition unless otherwise specified.

### EXAMPLE 1

### Preparation of Materials for the Cardio-Respiratory Sensor

Samples of PET/SPANDEX and nylon/SPANDEX fabrics in 1″×1″ dimensions were cut out and treated with O₂/Argon plasma. The PET/SPANDEX fabric is treated for 20 s, while the nylon/SPANDEX fabric is treated for 60 s.

To prepare, PEDOT only samples, the fabric sample was soaked with 95 wt % Clevios PH-1000 PEDOT:PSS and 5 wt % DMSO, annealed at 110° C. for 1 hr. The same procedure was repeated in case of multiple applications.

To prepare graphene/graphite only samples, 100 mg graphite was put in a 20 ml vial. 5 ml n-heptane was added in the vial followed by a brief bath sonication. It was tip sonicated for 15 min at 40% amplitude. 5 ml of DI water was added to the vial and it was tip sonicated at the liquid-liquid interface for 15 min at 40%. The vial was then filled with n-heptane to the top before the plasma treated spandex sample was added. It was bath sonicated for an hour and then the fabric was taken out and dried in the oven at 60° C. for 1 hr. The sample was weighed after being dried.

To prepare graphene/graphite and PEDOT samples, fabric coated with graphene/graphite following the previous procedure was soaked with 95 wt % Clevios PH-1000 PEDOT:PSS and 5 wt % DMSO, annealed at 110° C. for 1 hr. The sample was then weighed.

### EXAMPLE 2

### Method of Making All Fabric Cardio-Respiratory Sensor

This Example illustrates one embodiment of the method of making all fabric cardio-respiratory sensors. The method comprises the following steps.

First, a film or fabric, which is suitable for electro-activation, is stretched or expanded to create micro-voids within the structure of the fabric. The micro-voids are interleaved within the stretched fabric.

The stretched fabric is then subjected to corona electrical poling using conventional techniques to produce an electroactive fabric. Without being bound by a theory, it is believed that the stretched fabric become electroactive through charge separation as a result of micro-discharge or electrical breakdown within the pores of the fabric.

At least two conductive fabric electrodes are applied to the electroactive fabric to produce a sensor. At least one conductive fabric electrode is applied on either side of the electroactive fabric. A schematic diagram of the sensor is shown in FIG. 1.

The sensor thus produced generates an electrical signal in response to a force applied to the sensor. The signal generated by the sensor can readily be amplified by a current amplifier in conjunction with a low pass filter for signal conditioning and then fed into a commercially available digital or analog data acquisition apparatus for further signal processing or data storage.

It is noted that no battery is needed to generate an electrical signal in the sensor.

### EXAMPLE 3

### All-Fabric Sensor Comprising Open Pore Electroactive Fabric

This Example illustrates one embodiment of the all fabric sensor prepared according to the method described in Example 2.

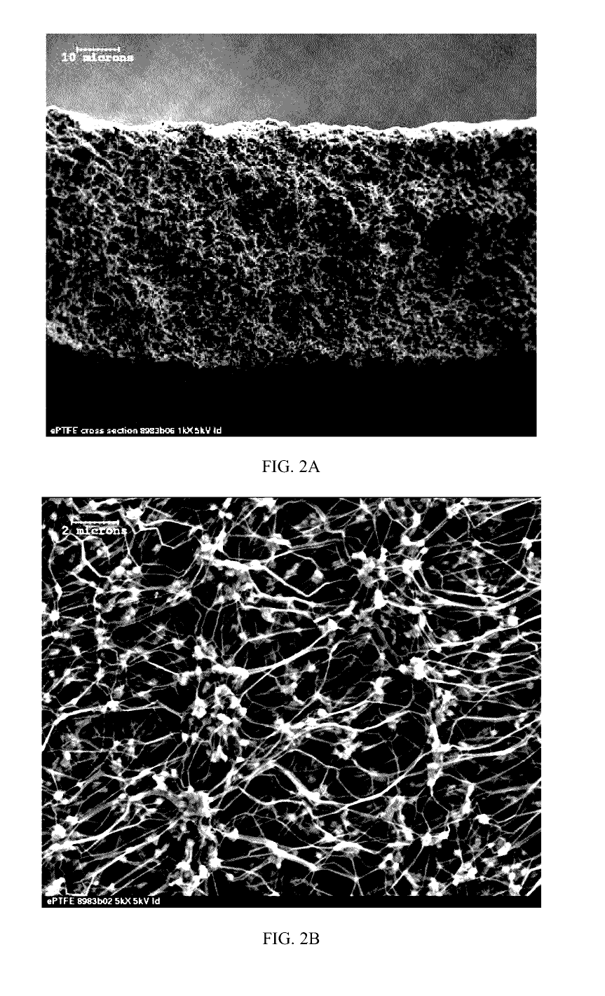

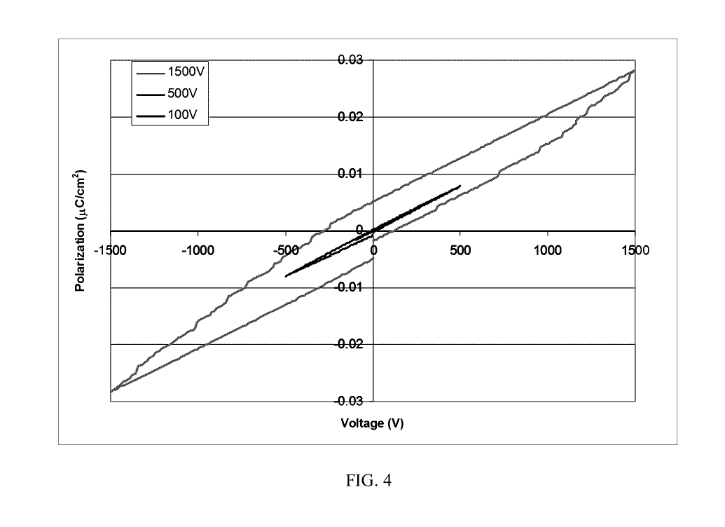

In this Example, a sensor is prepared according to the method of Example 1. The fabric used for electro-activation is polytetrafluoroethylene (PTFE). A cross-section view of the open pore electroactive fabric (expanded PTFE) is shown in FIG. 2A. A top view of the open pore electroactive fabric (expanded PTFE) is shown in FIG. 2B. FIG. 4 shows the D-E loop diagram for open pore electroactive fabric (Expanded PTFE) revealed by a modified Sewyer-Tower tester, indicating ferroelectric hysteresis behavior.

### EXAMPLE 4

### All-Fabric Sensor Comprising Closed Pore Electroactive Fabric

This Example illustrates another embodiment of the all-fabric sensor prepared according to the method described in Example 2.

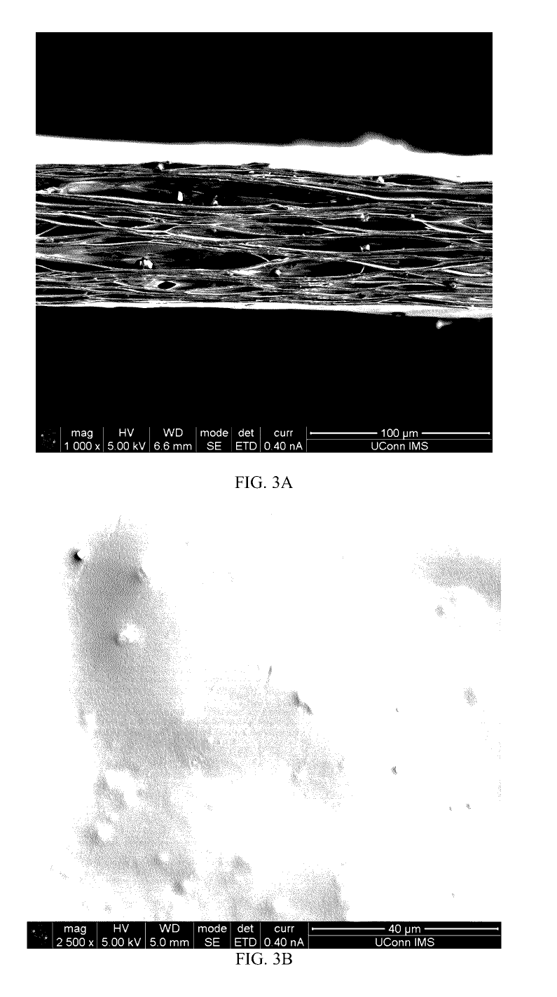

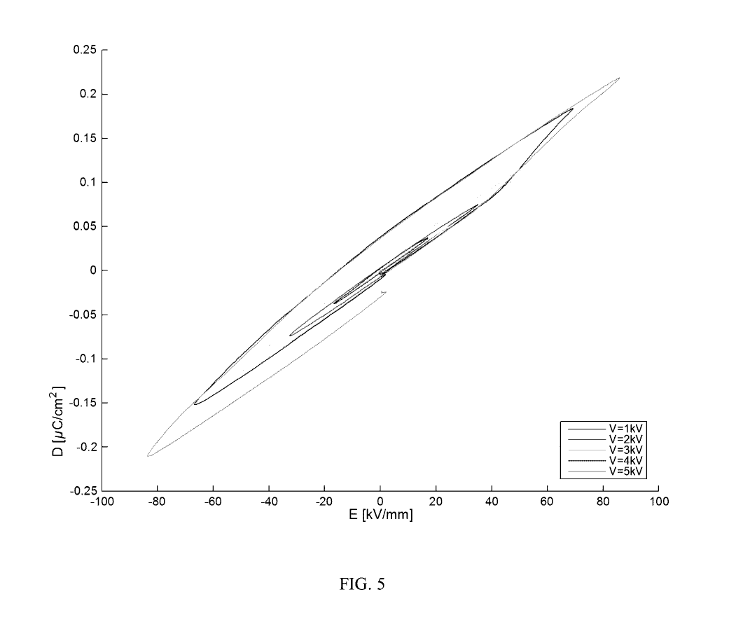

In this Example, a sensor is prepared according to the method of Example 1. The fabric used for electro-activation is a propylene fabric. A cross-section view of the closed pore electroactive fabric (propylene) is shown in FIG. 3A. A top view of the closed pore electroactive fabric (propylene) is shown in FIG. 3B. FIG. 5 shows D-E loop diagram for closed pore electroactive fabric (propylene) revealed by a modified Sewyer-Tower tester, indicating ferroelectric hysteresis behavior.

### EXAMPLE 5

### All-Fabric Sensor Comprising Synthetic Leather Electrodes

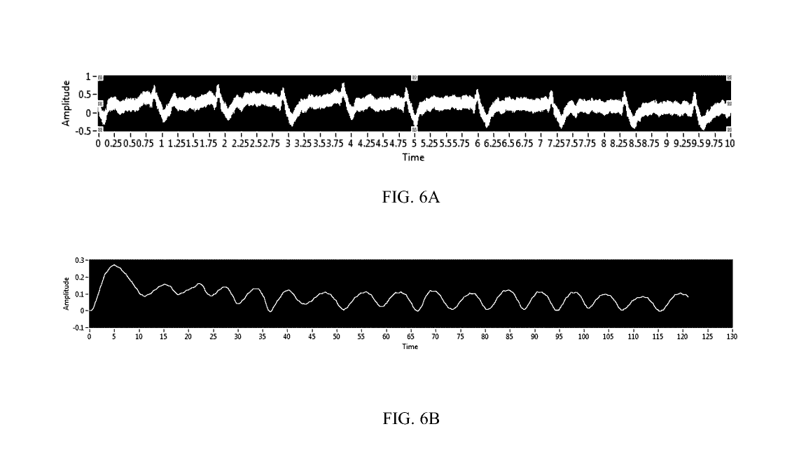

This Example illustrates another embodiment of the all-fabric sensor prepared according to the method described in Example 2. In this example, the conductive electrodes comprise PEDOT-PSS coated polyethyleneterephtaalate synthetic leather. FIG. 6A shows heart beat signal as recorded by the synthetic leather based sensor. FIG. 6B shows respiration rate signal as recorded by a synthetic leather based sensor.

### EXAMPLE 6

### All-Fabric Sensor Comprising Synthetic Spandex Electrodes

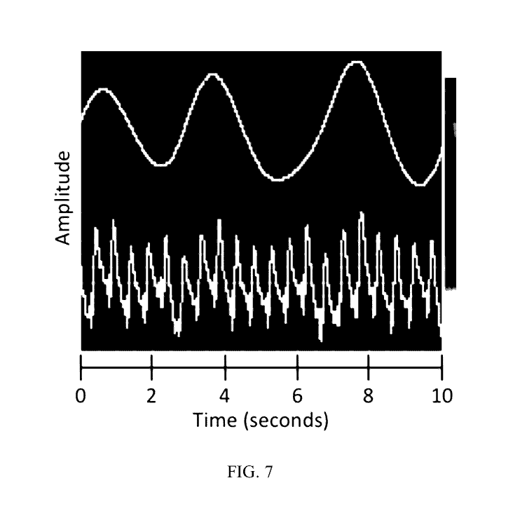

This Example illustrates another embodiment of the all-fabric sensor prepared according to the method described in Example 2. In this example, the conductive electrodes comprise PEDOT-PSS coated spandex. FIG. 7 shows respiratory rate and heart beat rate signals as recorded by a cardio-respiratory sensor using spandex electrodes (PEDOT-PSS coating).

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other. Each range disclosed herein constitutes a disclosure of any point or sub-range lying within the disclosed range.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

1. A sensor, comprising
at least two conductive electrodes; and
an electroactive region disposed between and in contact with the at least two conductive electrodes.

2. The sensor of claim 1, wherein the conductive electrodes are free of metals.

3. The sensor of claim 1, wherein the conductive electrodes comprise a fabric coated with a conductive organic polymer.

4. The sensor of claim 1, wherein the electroactive region comprises at least one layer of an electroactive fabric.

5. The sensor of claim 4, wherein the electroactive fabric comprises an open pore fabric.

6. The sensor of claim 5, wherein the open pore fabric is selected from the group consisting of propylene, polyethylene, fluorinated ethylene propylene, and PTFE.

7. The sensor of claim 4, wherein the electroactive fabric comprises a closed pore fabric.

8. The sensor of claim 7, wherein the electroactive fabric comprises a material selected from the group consisting of propylene, polyethylene, fluorinated ethylene propylene, and PTFE.

9. The sensor of claim 3, wherein the conductive electrodes comprise PEDOT-PSS coated polyethyleneterephtalate synthetic leather.

10. The sensor of claim 1, further comprising an apparatus in electrical communication with the at least two conductive electrodes wherein the apparatus is structured to detect a change in electrical potential between the at least two conductive electrodes.

11. The sensor of claim 1, wherein the apparatus comprises a charge amplifier and an ADC converter.

12. A method of making a sensor comprising
stretching a fabric to create micro-voids in the fabric;
subjected the fabric to electrical poling thereby forming an electroactive fabric;
attaching the electroactive fabric to at least two conductive electrodes such that the electroactive fabric is disposed between and in electrical contact with the at least two conductive electrodes.

13. The method of claim 12, wherein the electrical poling comprises a poling technique selected from the group consisting of corona electrical poling, short-circuit poling, and electron-beam irradiation poling.
